(19) United States
(12) Patent Application Publication
(10) Pub. No.: US 2024/0294483 A1
(43) Pub. Date: Sep. 5, 2024

(54) PROCESS FOR THE PREPARATION OF A LIQUID MIXTURE COMPRISING METHANOL, WATER AND HYDROGEN PEROXIDE

(71) Applicants: BASF SE, Ludwigshafen am Rhein (DE); Dow Global Technologies LLC, Midland, MI (US)

(72) Inventors: Andrei-Nicolae PARVULESCU, Ludwigshafen am Rhein (DE); Joaquim Henrique TELES, Ludwigshafen am Rhein (DE); Dominic RIEDEL, Ludwigshafen am Rhein (DE); Dylan SEGERS, Antwerp (BE); Massimo BERGAMO, Stade (DE); Franciscus Johannes Robertus VAN NEER, Hoek (NL); Meinolf WEIDENBACH, Stade (DE)

(21) Appl. No.: 18/572,209

(22) PCT Filed: Jun. 22, 2022
(86) PCT No.: PCT/EP2022/066967

§ 371 Date: Dec. 20, 2023

(30) Foreign Application Priority Data
Jun. 23, 2021 (EA) 21181263.1

(51) Int. Cl. C07D 301/12

(52) U.S. Cl. CPC C07D 301/12

(57) ABSTRACT

The present invention relates in a first aspect to a process for the preparation of a liquid mixture comprising methanol, water and hydrogen peroxide, the process comprising: (i) providing a feed stream comprising methanol, and a feed stream comprising an aqueous hydrogen peroxide solution; (ii) combining the feed stream comprising the methanol and the feed stream comprising the aqueous hydrogen peroxide solution provided according to (i) at a point in time t₁, so that a combined stream comprising methanol, hydrogen peroxide and water is obtained; (iii) filtrating the combined stream obtained according to (ii) at a point in time t₂, through a filtration device, thereby obtaining a liquid mixture comprising methanol, hydrogen peroxide and water; wherein the period of time between t₁ and t₂ is at least 8 seconds. In a second aspect, the invention relates to the use of a liquid mixture obtained or obtainable from the process of the first aspect for the preparation of propylene oxide. A third aspect of the invention is directed to a method for the preparation of propylene oxide comprising: (i) providing a feed stream comprising methanol, a feed stream comprising an aqueous hydrogen peroxide solution and a feed stream comprising propylene; (ii) combining the feed stream comprising the methanol and the feed stream comprising the aqueous hydrogen peroxide solution provided according to (i) at a point in time t₁, so that a combined stream comprising methanol, hydrogen peroxide and water is obtained; (iii) filtrating the combined stream obtained according to (ii) at a point in time t₂, through a filtration device, thereby obtaining a liquid mixture comprising methanol, hydrogen peroxide and water; (iv) combining the feed stream comprising propylene either with the combined stream obtained in (ii) or with the liquid mixture obtained in (iii), thereby obtaining a liquid mixture comprising methanol, hydrogen peroxide, water and propylene; (v) bringing the liquid mixture obtained according to (iv) in an epoxidation zone in contact with an epoxidation catalyst comprising a zeolitic material having a framework structure comprising Si, O, and Ti under epoxidation reaction conditions, thereby obtaining in the epoxidation zone a mixture comprising propylene oxide, methanol and water; wherein the period of time between t₁ and t₂ are at least 8 seconds. In a fourth aspect, the invention relates to propylene oxide, obtained or obtainable from the method according to the third aspect.

The present invention relates in a first aspect to a process for the preparation of a liquid mixture comprising methanol, water and hydrogen peroxide, the process comprising: (i) providing a feed stream comprising methanol, and a feed stream comprising an aqueous hydrogen peroxide solution; (ii) combining the feed stream comprising the methanol and the feed stream comprising the aqueous hydrogen peroxide solution provided according to (i) at a point in time t₁, so that a combined stream comprising methanol, hydrogen peroxide and water is obtained; (iii) filtrating the combined stream obtained according to (ii) at a point in time t₂, through a filtration device, thereby obtaining a liquid mixture comprising methanol, hydrogen peroxide and water; wherein the period of time between t₁ and t₂ is at least 8 seconds. In a second aspect, the invention relates to the use of a liquid mixture obtained or obtainable from the process of the first aspect for the preparation of propylene oxide. A third aspect of the invention is directed to a method for the preparation of propylene oxide comprising: (i) providing a feed stream comprising methanol, a feed stream comprising an aqueous hydrogen peroxide solution and a feed stream comprising propylene; (ii) combining the feed stream comprising the methanol and the feed stream comprising the aqueous hydrogen peroxide solution provided according to (i) at a point in time t₁, so that a combined stream comprising methanol, hydrogen peroxide and water is obtained; (iii) filtrating the combined stream obtained according to (ii) at a point in time t₂, through a filtration device, thereby obtaining a liquid mixture comprising methanol, hydrogen peroxide and water; (iv) combining the feed stream comprising propylene either with the combined stream obtained in (ii) or with the liquid mixture obtained in (iii), thereby obtaining a liquid mixture comprising methanol, hydrogen peroxide, water and propylene; (v) bringing the liquid mixture obtained according to (iv) in an epoxidation zone in contact with an epoxidation catalyst comprising a zeolitic material having a framework structure comprising Si, O, and Ti under epoxidation reaction conditions, thereby obtaining in the epoxidation zone a mixture comprising propylene oxide, methanol and water; wherein the period of time between t₁ and t₂ are at least 8 seconds. In a fourth aspect, the invention relates to propylene oxide, obtained or obtainable from the method according to the third aspect.

Olefin oxides such as propylene oxide (PO) are important intermediates in the chemical industry. Traditionally, PO is produced via the chlorohydrin process, which is still in use today, as well as the oxirane method. The development of catalysts based on zeolitic materials having a framework structure comprising Si, O, and Ti, such as titanium silicalite-1, together with the improved availability of large quantities of hydrogen peroxide enabled the large-scale implementation of the co-product-free synthesis of olefin oxides from the corresponding olefins by reaction with hydrogen peroxide—for propylene oxide, this is the so called HPPO technology. This new process enables olefin oxides such as PO to be produced with excellent yields and selectivities.

The liquid phase epoxidation of olefins with hydrogen peroxide catalyzed by a fixed bed titanium silicalite catalyst is known. The reaction can be carried out in different solvents, wherein the most prominent solvents are acetonitrile and methanol. Epoxidation, preferably continuous epoxidation, of the olefin is achieved by passing a mixture comprising the olefin, hydrogen peroxide and the solvent through a fixed bed of the epoxidation catalyst.

Especially when using methanol as the solvent and using an aqueous hydrogen peroxide solution, which was made by an anthraquinone process, i.e. a liquid mixture comprising methanol, water and hydrogen peroxide, it is known that precipitates form, presumably due to the presence of stabilizers and metal impurities in the aqueous hydrogen peroxide solution. The precipitates can result in deposits on the catalyst or, if the reactants are transferred into the reactor through a liquid distributor, deposits can form or accumulate at the orifices of such distributors and blocking of the orifices by the deposits can lead to a maldistribution of feed in the reactor. The deposits on the catalyst are not removed by usual catalyst regeneration procedures like washing with solvent or heating, the same applies in principle to the blocked orifices of a liquid distributor.

It is also known that the precipitation of such deposits could be avoided by using simple means such as a filter, see, for example, EP 3 380 459 B1. However, using a filter imposes new problems, for example, in view of the positioning of the filter relative to where the precipitates initially forms in the liquid mixture.

It was thus an object of the present invention to provide an advantageous process for the preparation of a liquid mixture comprising methanol, water and hydrogen peroxide.

In a first aspect, the present invention thus relates to a process for the preparation of a liquid mixture comprising methanol, water and hydrogen peroxide, the process comprising
(i) providing a feed stream comprising methanol, and a feed stream comprising an aqueous hydrogen peroxide solution;
(ii) combining the feed stream comprising the methanol and the feed stream comprising the aqueous hydrogen peroxide solution provided according to (i) at a point in time t₁, so that a combined stream comprising methanol, hydrogen peroxide and water is obtained;
(iii) filtrating the combined stream obtained according to (ii) at a point in time t₂, through a filtration device, thereby obtaining a liquid mixture comprising methanol, hydrogen peroxide and water;
wherein the period of time between t₁ and t₂ is at least 8 seconds.

The period of time between t₁ and t₂ is also called “residence time” (t₂-t₁) and means the time span during which at least the reaction feed stream comprising the methanol and the reaction feed stream comprising the aqueous hydrogen peroxide solution are in contact with each other before the combined stream from (ii) reaches the filtration device used in (iii).

When mixing aqueous hydrogen peroxide with an organic solvent such as methanol, it was shown that the precipitate formation required a certain amount of time after the aqueous hydrogen peroxide came in contact with the methanol. It could be shown that the residence time of a combined stream comprising methanol and aqueous hydrogen peroxide solution before coming in contact with a filter had to be at least 8 seconds. If the residence time was less than 8 seconds, the precipitate had not enough time to form in the feed tube before the filter but moreover formed after the filter, thus impairing the catalyst's performance and, consequently, the performance of the epoxidation reaction.

The residence time can be easily calculated from the volume present between the position, where the reaction feed stream comprising the methanol and the reaction feed stream comprising the aqueous hydrogen peroxide solution are brought in contact with each other, and the position, where the filtration device is located and the volumetric flow rate, especially the volumetric flow rate of the combined stream obtained according to (ii). If, for example, the combined stream flows through a pipe before it reaches the filtration device, the volume to be considered is the pipe volume from the position, where the feed stream comprising the aqueous hydrogen peroxide solution and the feed comprising the methanol are brought in contact with each other, to the filtration device. In case the filtration device is comprised in a filtration unit, the volume is the volume of the pipe from the position, where the feed stream comprising the aqueous hydrogen peroxide solution and the feed comprising the methanol are brought in contact with each other+the volume within the filtration unit up to the filtration device. The parameters volume and volumetric flow rate can be adjusted by a person skilled in the art in order to establish a suitable residence time. The residence time is in any case a longer time span than the required mixing time τ, which is the time, which, after the reaction feed stream comprising the methanol and the reaction feed stream comprising the aqueous hydrogen peroxide solution are brought in contact with each other at the point in time t₁, is required until these reaction feeds are completely intermixed with each other so that the combined stream comprising methanol, hydrogen peroxide and water is obtained (t₂−t₁>τ), for example, the combined stream flows through a pipe, the mixing time t is defined as in formula (I):

$$τ = \frac{( \frac{π {D}^{2}}{4} ) L}{( {F}_{1} + {F}_{2} )} \qquad ( I )$$

wherein
D is the inner diameter of the pipe;
L is the length of the pipe from the point where the reaction feed stream comprising the methanol and the reaction feed stream comprising the aqueous hydrogen peroxide solution are brought in contact with each other up to the point, where these reaction feed streams are intermixed;
F₁ is the flow rate of the reaction feed stream comprising the methanol;
F₂ is the flow rate of the reaction feed stream comprising the aqueous hydrogen peroxide solution.

It goes without saying that the mixing time t can be influenced by using, for example, a mixing device within the pipe-preferably downstream from the point where the two reaction feeds are brought in contact with each other and upstream from the point where the filtration device is located, since this would have an impact on L. “Downstream” and “upstream” are clear to a skilled person with respect to the flow direction of the combined stream towards the filtration device, and, subsequently, any following epoxidation zone. Knowing the geometry of the mixing section it is easily possible using computation fluid dynamics (CFD) to estimate the mixing time. This is a method well known in the state of the art.

The liquid mixture comprising methanol, hydrogen peroxide and water obtained in (iii) is preferably essentially free of undissolved solids, which more preferably means that at least 95 weight-%, more preferably at least 98 weight-%, more preferably at least 99 weight-%, more preferably at least 99.9 weight-% of the liquid mixture comprising methanol, hydrogen peroxide and water obtained in (iii) are liquid, each based on the total weight of the liquid mixture.

According to a preferred embodiment, the process for the preparation of a liquid mixture comprising methanol, water and hydrogen peroxide comprises
(iv) adding to the feed stream comprising methanol a further feed stream comprising propylene before (ii) or adding to the liquid mixture obtained in (iii) a further feed stream comprising propylene,
thereby obtaining a liquid mixture comprising methanol, hydrogen peroxide, water and propylene.

According to a preferred embodiment, the process for the preparation of a liquid mixture comprising methanol, water and hydrogen peroxide comprises
(i) providing a feed stream comprising methanol and a feed stream comprising an aqueous hydrogen peroxide solution;
(ii-a) combining the feed stream comprising the methanol and the feed stream comprising the aqueous hydrogen peroxide solution provided according to (i) at a point in time t₁, so that a combined stream comprising methanol, hydrogen peroxide and water is obtained;
(iii-a) filtrating the combined stream obtained according to (ii-a) at a point in time t₂,
through a filtration device, thereby obtaining a liquid mixture comprising methanol, hydrogen peroxide and water;
(iv-a) adding to the liquid mixture obtained in (iii-a) a further feed stream comprising propylene, thereby obtaining a liquid mixture comprising methanol, hydrogen peroxide, water and propylene;
wherein the period of time between t₁ and t₂ are at least 8 seconds.

According to this embodiment, step (iv-a) is either carried out in co-current mode or in counter current mode. For example, if the addition of step (iv-a) is done within a vessel, for example, a reactor which comprises an epoxidation zone, the liquid mixture obtained in (iii-a) enters the reactor from one direction (either side or top/bottom) and the further feed stream comprising propylene enters the reactor either from the same direction (either side or top/bottom) or from an another, preferably an opposite, direction. In a preferred constellation wherein a vertically arranged reactor is used, the liquid mixture obtained in (iii-a) enters the reactor from the top and the further feed stream comprising propylene enters the reactor from the bottom.

According to an alternative preferred embodiment, the process for the preparation of a liquid mixture comprising methanol, water and hydrogen peroxide comprises
(i) providing a feed stream comprising methanol and a feed stream comprising an aqueous hydrogen peroxide solution;
(ii-b) combining the feed stream comprising the methanol with a further feed stream comprising propylene thereby obtaining a stream comprising methanol and propylene;
(iii-b) combining the stream comprising methanol and propylene obtained in (ii-b) and the feed stream comprising the aqueous hydrogen peroxide solution at a point in time t₁, so that a combined stream comprising methanol, hydrogen peroxide, water and propylene is obtained;
(iv-b) filtrating the combined stream obtained according to (iii-b) at a point in time t₂ through a filtration device, thereby obtaining a liquid mixture comprising methanol, hydrogen peroxide, water and propylene;
wherein the period of time between t₁ and t₂ are at least 8 seconds.

According to this embodiment, if the liquid mixture comprising methanol, hydrogen peroxide, water and propylene is further transferred to a vessel, for example, a reactor which comprises an epoxidation zone, the liquid mixture comprising methanol, hydrogen peroxide, water and propylene obtained in (iv-b) enters the reactor from any suitable direction (either side or top/bottom). In a preferred constellation wherein a horizontally arranged reactor is used, the liquid mixture obtained in (iv-b) enters the reactor from a side. Preferably, a constellation with a vertically arranged reactor is used, wherein the liquid mixture obtained in (iv-b) enters the reactor from the bottom. More preferably, a constellation with a vertically arranged reactor is used, wherein the liquid mixture obtained in (iv-b) enters the reactor from the bottom and flow direction in the reactor and through the epoxidation zone is from bottom to top.

In a preferred embodiment of process for the preparation of a liquid mixture comprising methanol, water and hydrogen peroxide, the period of time between t₁ and t₂ is a period in the range of from 8 seconds to 5 hours, more preferably in the range of from 8 seconds to 4 hours, more preferably in the range of from 8 seconds to 3 hours, more preferably in the range of from 8 seconds to 2 hours, more preferably in the range of from 8 seconds to 1 hour. More preferably, the period of time between 1 and t₂ are at least 10 seconds, preferably at least 20 seconds, more preferably at least 25 seconds. More preferably, the period of time between t₁ and t₂ is a period in the range of from 10 seconds to 5 hours, more preferably in the range of from 10 seconds to 4 hours, more preferably in the range of from 10 seconds to 3 hours, more preferably in the range of from 10 seconds to 2 hours, more preferably in the range of from 10 seconds to 1 hour. More preferably, the period of time between t₁ and t₂ is a period in the range of from 20 seconds to 5 hours, more preferably in the range of from 20 seconds to 4 hours, more preferably in the range of from 20 seconds to 3 hours, more preferably in the range of from 20 seconds to 2 hours, more preferably in the range of from 20 seconds to 1 hour. More preferably, the period of time between t₁ and t₂ is a period in the range of from 25 seconds to 5 hours, more preferably in the range of from 25 seconds to 4 hours, more preferably in the range of from 25 seconds to 3 hours, more preferably in the range of from 25 seconds to 2 hours, more preferably in the range of from 25 seconds to 1 hour.

Regarding the preferred embodiment indicated above comprising steps (i), (ii-a), (iii-a) and (iv-a), the period of time between t₁ and t₂ is preferably a period in the range of from 8 seconds to 5 hours, more preferably in the range of from 8 seconds to 4 hours, more preferably in the range of from 8 seconds to 3 hours, more preferably in the range of from 8 seconds to 2 hours, more preferably in the range of from 8 seconds to 1 hour. More preferably, the period of time between t₁ and t₂ are at least 10 seconds, preferably at least 20 seconds, more preferably at least 25 seconds. More preferably, the period of time between t₁ and t₂ is a period in the range of from 10 seconds to 5 hours, more preferably in the range of from 10 seconds to 4 hours, more preferably in the range of from 10 seconds to 3 hours, more preferably in the range of from 10 seconds to 2 hours, more preferably in the range of from 10 seconds to 1 hour. More preferably, the period of time between t₁ and t₂ is a period in the range of from 20 seconds to 5 hours, more preferably in the range of from 20 seconds to 4 hours, more preferably in the range of from 20 seconds to 3 hours, more preferably in the range of from 20 seconds to 2 hours, more preferably in the range of from 20 seconds to 1 hour. More preferably, the period of time between t₁ and t₂ is a period in the range of from 25 seconds to 5 hours, more preferably in the range of from 25 seconds to 4 hours, more preferably in the range of from 25 seconds to 3 hours, more preferably in the range of from 25 seconds to 2 hours, more preferably in the range of from 25 seconds to 1 hour.

Regarding the preferred embodiment indicated above comprising steps (i), (ii-b), (iii-b) and (iv-b), the period of time between t₁ and t₂ is preferably a period in the range of from 8 seconds to 5 hours, more preferably in the range of from 8 seconds to 4 hours, more preferably in the range of from 8 seconds to 3 hours, more preferably in the range of from 8 seconds to 2 hours, more preferably in the range of from 8 seconds to 1 hour. More preferably, the period of time between t₁ and t₂ are at least 10 seconds, preferably at least 20 seconds, more preferably at least 25 seconds. More preferably, the period of time between t₁ and t₂ is a period in the range of from 10 seconds to 5 hours, more preferably in the range of from 10 seconds to 4 hours, more preferably in the range of from 10 seconds to 3 hours, more preferably in the range of from 10 seconds to 2 hours, more preferably in the range of from 10 seconds to 1 hour. More preferably, the period of time between t₁ and t₂ is a period in the range of from 20 seconds to 5 hours, more preferably in the range of from 20 seconds to 4 hours, more preferably in the range of from 20 seconds to 3 hours, more preferably in the range of from 20 seconds to 2 hours, more preferably in the range of from 20 seconds to 1 hour. More preferably, the period of time between t₁ and t₂ is a period in the range of from 25 seconds to 5 hours, more preferably in the range of from 25 seconds to 4 hours, more preferably in the range of from 25 seconds to 3 hours, more preferably in the range of from 25 seconds to 2 hours, more preferably in the range of from 25 seconds to 1 hour.

The upper limit of the period of time between t₁ and t₂ of 5 hours, preferably 4 hours, more preferably 3 hours, more preferably 2 hours, more preferably 1 hour is essential since it has been observed that with longer periods of time, precipitate forms in the equipment used such as the feed line, through which the combined stream obtained according to (ii-a) or (ii-b), preferably the combined stream obtained according to (ii.a), flows towards the filtration device, wherein said precipitate negatively influences flow and distribution on the filtration device of the combined stream. It was further found that precipitate in the feed line has a negative effect on the hydrogen peroxide contained in the combined stream in that it causes/catalyses an increased decomposition of the hydrogen peroxide, which in turn causes severe safety issues and has a negative impact on yield and selectivity of the final propylene oxide. The more of the feed line length before the filter is covered with precipitate, the more contact time results for the hydrogen peroxide streaming over the precipitate and the more pronounced is the decomposition. The increased decomposition of hydrogen peroxide in the presence of precipitate was also proven by differential scanning calorimetry (DSC).

The feed stream comprising methanol, the feed stream comprising an aqueous hydrogen peroxide solution and also the (further) feed stream comprising propylene each have a temperature in the range of from 0 to 100° C., preferably in the range of from 10 to 80° C., more preferably in the range of from 15 to 60° C. The same temperature ranges, preferred temperature ranges and more preferred temperature ranges, apply for the combined stream obtained in (ii), (ii-a), (ii-b) and (iii-b), the liquid mixture obtained in (iii), (iii-a) or (iv-a).

### Hydrogen Peroxide

In a preferred embodiment of process for the preparation of a liquid mixture comprising methanol, water and hydrogen peroxide, the hydrogen peroxide is provided as aqueous hydrogen peroxide solution, which has a total organic carbon content (TOC) in the range of from 100 to 800 mg per kg hydrogen peroxide comprised in the aqueous hydrogen peroxide solution, more preferably in the range of from 120 to 750 mg per kg hydrogen peroxide comprised in the aqueous hydrogen peroxide solution, more preferably in the range of from 150 to 700 mg per kg hydrogen peroxide comprised in the aqueous hydrogen peroxide solution, determined according to DIN EN 1484 (April 2019). Preferably, the aqueous hydrogen peroxide solution has a pH in the range of from 0 to 3.0, more preferably in the range of from 0.1 to 2.5, more preferably in the range of from 0.5 to 2.3, determined with a pH sensitive glass electrode according to CEFIC PEROXYGENS H2O2 AM-7160 standard (2003). Preferably, the aqueous hydrogen peroxide solution comprises from 20 to 85 weight-%, more preferably from 30 to 75 weight-%, more preferably from 40 to 70 weight-% of hydrogen peroxide, relative to the total weight of the aqueous hydrogen peroxide solution.

In a preferred embodiment of process for the preparation of a liquid mixture comprising methanol, water and hydrogen peroxide, the hydrogen peroxide, preferably the aqueous hydrogen peroxide solution, is obtained or obtainable from an anthraquinone process.

The aqueous hydrogen peroxide solution from an anthraquinone process is obtained as crude hydrogen peroxide solution by extraction of a mixture which results from an anthraquinone process (see, for example, Ullmann's Encyclopedia of Industrial Chemistry, 5th edition, volume A 13 (1989) pages 443-466), wherein a solution of an anthraquinone is used containing an alkyl group preferably having from 2 to 10 carbon atoms, more preferred a 2-6 carbon atoms, more preferred 2, 5 or 6 carbon atoms, and where the solvent used usually consists of a mixture of at least two different solvents. Preferably, mixtures of two solvents or mixtures of three solvents are used. Preferably, none of the solvents used in the anthraquinone process is a nitrogen containing substance. This solution of the anthraquinone is usually referred to as the working solution. In this process, the hydrogen peroxide formed in the course of the anthraquinone process is generally separated by extraction from the respective working solution after a hydrogenation/re-oxidation cycle. Said extraction can be performed preferably with essentially pure water, and the crude aqueous hydrogen peroxide solution is obtained. It is generally possible to further purify and/or concentrate the thus obtained crude aqueous hydrogen peroxide solution by distillation. It is possible to use crude aqueous hydrogen peroxide solution which has not been subjected to purification and/or concentration by distillation and it is also possible to use an aqueous hydrogen peroxide solution which has been subjected to purification and/or concentration by distillation. Further, it is generally possible to subject the crude aqueous hydrogen peroxide solution to a further extraction stage wherein a suitable extracting agent, preferably an organic solvent is used. More preferred, the organic solvent used for this further extraction stage is the same solvent which is used in the anthraquinone process. Preferably the extraction is performed using just one of the solvents in the working solution and most preferably using just the most nonpolar solvent of the working solution. In case the crude aqueous hydrogen peroxide solution is subjected to such further extraction stage, a so-called crude washed hydrogen peroxide solution is obtained. According to a preferred embodiment of the present invention, the crude washed hydrogen peroxide solution is used as the aqueous hydrogen peroxide solution. The production of a crude solution is described, for example, in European patent application EP 1 122 249 A1. As to the term “essentially pure water”, reference is made to paragraph 10, page 3 of EP 1 122 249 A1 which is incorporated by reference. The hydrogen peroxide can also be treated to remove trace metals, for example, as described in the WO 2015/049327 A1 before use.

In a preferred embodiment of process for the preparation of a liquid mixture comprising methanol, water and hydrogen peroxide, the aqueous hydrogen peroxide solution contains in the range of from 0.1 to 10 mg of non-alkali metal cations per kg of hydrogen peroxide, preferably in the range of from 0.25 to 5 mg of non-alkali metal cations per kg of hydrogen peroxide, wherein the non-alkali metal cations are preferably selected from the group consisting of cations of Si, Fe, Ni, Mn, Al, Cr, Pd, Ca, Mg and mixtures of two or more of these metal cations. Preferably, the aqueous hydrogen peroxide solution is stabilized with a stabilizer selected from the group consisting of phosphoric acid, pyrophosphoric acid, nitric acid, dialkali hydrogenphosphate, alkali dihydrogen phosphate, dialkali pyrophosphate, tetraalkali pyrophosphate, ammonium nitrate, alkali nitrate and mixtures of two or more of these stabilizers, wherein the alkali metal is preferably sodium or potassium, more preferably sodium. Preferably, the aqueous hydrogen peroxide solution comprises in the range of from 0 to 0.010 weight-% sodium cations (0 to 100 weight-ppm), and in the range of from 0.001 to 0.050 weight-% (10 to 500 weight-ppm), more preferably in the range of from 0.005 to 0.025 weight-% (50 to 250 weight-ppm) phosphorus, calculated as phosphate (PO₄³⁻), each based on the total weight of the aqueous hydrogen peroxide solution.

### Water

No restrictions exist regarding the water used for the liquid mixture. It is conceivable to use, for example, water which is treated with NH₃ but water not having been treated with NH₃ can also be used. Preferably deionized water is used for the reaction mixture. The deionized water can be obtained using ion-exchangers of using condensate. Typical grades of deionized water are defined in ISO 3696 of 1987 and all grades described there can be used within the scope of this invention. The water may additionally contain traces of corrosion inhibiting additives like ammonia, hydrazine or hydroxylamine in which case it should have a pH value in the range of 7 to 9 (determined with a pH sensitive glass electrode according to CEFIC PEROXYGENS H2O2 AM-7160 standard (2003)). Preferably, the water used does not contain corrosion inhibiting additives.

As indicated above, it is common knowledge that precipitates form if an aqueous hydrogen peroxide solution from an anthraquinone process and/or which comprises stabilizers is combined with an organic solvent such as methanol. Without being bound to this theory, it is plausible to assume that the precipitate comprises a less soluble crystallization product of one or more of the above-described phosphorous containing anions and one or more metal cations, wherein “metal cations” comprises the above-described non-alkali metal cations and alkali cations, wherein the alkali cations are preferably Na⁺ and/or K⁺.

Methanol is the solvent used in the above-described process. However, a person skilled in the art understands that the process described herein above is also suitable for other organic solvents, such as organic epoxidation solvents, for example selected from the group consisting of alcohol, acetonitrile, propionitrile and mixtures of two or more thereof; more preferably selected from the group consisting of branched or unbranched C1 to C5 mono alcohol, acetonitrile and mixtures of branched or unbranched C1 to C5 mono alcohol and acetonitrile, more preferably tert. butanol, acetonitrile and mixtures of tert. butanol and acetonitrile.

### Ratios in Combined Stream Prior to Filtration

According to another preferred embodiment of the process for the preparation of a liquid mixture comprising methanol, water and hydrogen peroxide, the weight ratio of methanol:hydrogen peroxide (w/w) in the combined stream obtained in (ii), (ii-a) or (ii-b) is in the range of from 15:1 to 5:1, more preferably in the range of from 12:1 to 6:1, more preferably in the range of from 12:1 to 9:1 or in the range of from 8:1 to 6:1. In the combined stream obtained in (ii-b), the weight ratio of propylene:hydrogen peroxide (w/w) is preferably in the range of from 1:1 to 5:1, more preferably in the range of from 1:1 to 2:1 or in the range of from 3:1 to 5:1 and the weight ratio of methanol:propylene (w/w) is preferably in the range of from 10:1 to 1:0.1, more preferably in the range of from 9:1 to 1:1, more preferably in the range of from 9:1 to 7:1 or in the range of from 1.5:1 to 1:1.

### Ratios in Liquid Mixture

According to another preferred embodiment of the process for the preparation of a liquid mixture comprising methanol, water and hydrogen peroxide, the weight ratio of propylene:hydrogen peroxide (w/w) in the liquid mixture, which is obtained in (iv), (iv-a) or (iii-b), is in the range of from 1:1 to 5:1, preferably in the range of from 1:1 to 2:1 or in the range of from 3:1 to 5:1. Preferably, the weight ratio of methanol:hydrogen peroxide (w/w) in the liquid mixture, which is obtained in (iv), (iv-a) or (iii-b), is in the range of from 15:1 to 5:1, more preferably in the range of from 12:1 to 6:1, more preferably in the range of from 12:1 to 9:1 or in the range of from 8:1 to 6:1. Preferably, the weight ratio of methanol:propylene (w/w) in the liquid mixture, which is obtained in (iv), (iv-a) or (iii-b), is in the range of from 10:1 to 1:0.1, more preferably in the range of from 9:1 to 1:1, more preferably in the range of from 9:1 to 7:1 or in the range of from 1.5:1 to 1:1.

### Filtration Device

According to another preferred embodiment of the process for the preparation of a liquid mixture comprising methanol, water and hydrogen peroxide, the filtration device according to (iii), (iii-a) and/or (iii-b) comprises a filter, preferably a filter selected from the group consisting of pleated filter, CUNO™ type filter and mixed forms of these filter types. The filter is preferably operated with a specific loading, which is the quotient of the volume flow rate per filter area in the range of from 0.05×10⁻⁴ to 500×10⁻⁴ m/s, more preferably in the range of from 0.5×10⁻⁴ to 50×10⁻⁴ m/s, more preferably in the range of from 1×10⁻⁴ to 10×10⁻⁴ m/s. The volume flow rate is the sum of the mass flow rates of all streams forming the respective combined stream divided by the density of the respective combined stream according to (ii), (ii-a) or (ii-b).

The filter preferably has an nominal rating in the range of from 0.1 to 50 μm, more preferably in the range of from 0.5 to 50 μm, more preferably in the range of from 1 to 25 μm, more preferably in the range of from 1 to 10 μm. in some embodiments, the filter is selected from the group consisting of 3M 700B-HF40PP005K01 (nominal rating 5 μm), EFC PF-1306 (nominal rating 5 μm), PALL PRMMFS740H010V (nominal rating 10 μm), PALL MARKSMAN POLYFINE 740 XLD (nominal rating 5 μm), PALL PFTM5-740E (nominal rating 5 μm), 3M HFM60PPNO5D (nominal rating 5 μm), 3M 744B 740K40PP005D1 (nominal rating 5 μm), PARKER MAXGUARD MXGP200-40-E-SM (nominal rating 20 μm), PALL J200 (PRMMFS740TSJ10UX) (nominal rating 5 μm); more preferably from the group consisting of 3M 700B-HF40PP005K01 (nominal rating 5 μm), PARKER MAXGUARD MXGP200-40-E-SM (nominal rating 20 μm), and PALL J200 (PRMMFS740TSJ10UX). A nominal rating indicates the filter's ability to prevent the passage of a minimum percentage of solid particles greater than the nominal rating's stated micron size, wherein “size” means the average diameter. The nominal rating values indicated herein preferably mean that the filter prevents 90% of all particles having the indicated average diameter or having a larger average diameter from passing through. In case of unsymmetrical particles, the average diameter is related to the particle's largest diameter.

The filter area (the area of filtration) of the filtration device preferably means the filter area per technical reactor and is preferably in the range of from 0.01 to 10000 m², more preferably in the range of from 0.1 to 1000 m², more preferably in the range of from 1 to 250 m². Preferably, the filtration device comprises a housing in which one or more filtration cartridges, each cartridge comprising a filter, are arranged. In case that more than one cartridge is used, the filter area per technical reactor can be divided between the cartridges.

According to another preferred embodiment of the process for the preparation of a liquid mixture comprising methanol, water and hydrogen peroxide, the filter is a pleated filter, preferably a pleated filter wherein the pleats are at least partially laid over each other. Pleated filters with a so called laid over construction are known to the skilled person, for example, pleated filters with a laid over pleat geometry are commercially available from Pall Corporation, US. The laid over pleat geometry significantly increases the area of filtration and allows for a uniform flow distribution compared to traditional fan pleats. The pleated filter is preferably surrounded by external supporting elements. External supporting elements are more preferably aligned along an outer wall of the filter. For example, in case the filter is comprised in a filtration unit which has the shape of a tube with the pleated filter inside of the tube, preferably the laid over pleated filter being arranged between an outer wall and an inner wall of the tube, straps are arranged so that they surround the tube on its outer wall and essentially crossdirectional with respect to the tube's axis. “Essentially crossdirectional” means that the straps are arranged in an angle in the range of from 70 to 110° with respect to the tube's axis. Thus, the straps are advantageous in order to avoid damage when the filter is backflushed.

According to another preferred embodiment of the process for the preparation of a liquid mixture comprising methanol, water and hydrogen peroxide, the filter material comprises, preferably consists of, polypropylene or polyethylene, preferably polypropylene (PP).

### 2^{nd} Aspect—Use of Liquid Mixture

In a second aspect, the invention relates to the use of a liquid mixture obtained or obtainable from the process as disclosed above for the first aspect for the preparation of propylene oxide.

### 3^{rd} Aspect—Method for the Preparation of Propylene Oxide

A third aspect of the invention is related to a method for the preparation of propylene oxide comprising
(i) providing a feed stream comprising methanol, a feed stream comprising an aqueous hydrogen peroxide solution and a feed stream comprising propylene;
(ii) combining the feed stream comprising the methanol and the feed stream comprising the aqueous hydrogen peroxide solution provided according to (i) at a point in time t₁, so that a combined stream comprising methanol, hydrogen peroxide and water is obtained;
(iii) filtrating the combined stream obtained according to (ii) at a point in time t₂, through a filtration device, thereby obtaining a liquid mixture comprising methanol, hydrogen peroxide and water;
(iv) combining the feed stream comprising propylene either with the combined stream obtained in (ii) or with the liquid mixture obtained in (iii), thereby obtaining a liquid mixture comprising methanol, hydrogen peroxide, water and propylene;
(v) bringing the liquid mixture obtained according to (iv) in an epoxidation zone in contact with an epoxidation catalyst comprising a zeolitic material having a framework structure comprising Si, O, and Ti under epoxidation reaction conditions, thereby obtaining in the epoxidation zone a mixture comprising propylene oxide, methanol and water;
wherein the period of time between t₁ and t₂ are at least 8 seconds.

According to a preferred embodiment of method for the preparation of propylene oxide, the method comprises
(ii) providing a feed stream comprising methanol and a feed stream comprising an aqueous hydrogen peroxide solution;
(ii-a) combining the feed stream comprising the methanol and the feed stream comprising the aqueous hydrogen peroxide solution provided according to (i) at a point in time t₁, so that a combined stream comprising methanol, hydrogen peroxide and water is obtained;
(iii-a) filtrating the combined stream obtained according to (ii-a) at a point in time t₂, through a filtration device, thereby obtaining a liquid mixture comprising methanol, hydrogen peroxide and water;
(iv-a) adding to the liquid mixture obtained in (iii-a) a further feed stream comprising propylene, thereby obtaining a liquid mixture comprising methanol, hydrogen peroxide, water and propylene;
(v-a) bringing the liquid mixture obtained according to (iv-a) in an epoxidation zone in contact with an epoxidation catalyst comprising a zeolitic material having a framework structure comprising Si, O, and Ti under epoxidation reaction conditions, thereby obtaining in the epoxidation zone a mixture comprising propylene oxide, methanol and water;
wherein the period of time between t₁ and t₂ are at least 8 seconds.

According to this embodiment, step (iv-a) is either carried out in co-current mode or in counter current mode. For example, the addition of step (iv-a) is done within a reactor which comprises the epoxidation zone, the liquid mixture obtained in (iii-a) enters the reactor from one direction (either side or top/bottom) and the further feed stream comprising propylene enters the reactor either from the same direction (either side or top/bottom) or from an another, preferably an opposite, direction. In a preferred constellation wherein a vertically arranged reactor is used, the liquid mixture obtained in (iii-a) enters the reactor from the top and the further feed stream comprising propylene enters the reactor from the bottom.

According to an alternative preferred embodiment of method for the preparation of propylene oxide, the method comprises
(ii) providing a feed stream comprising methanol and a feed stream comprising an aqueous hydrogen peroxide solution;
(iv-b) combining the feed stream comprising the methanol with a further feed stream comprising propylene thereby obtaining a stream comprising methanol and propylene;
(ii-b) combining the stream comprising methanol and propylene obtained in (iv-b) and the feed stream comprising the aqueous hydrogen peroxide solution at a point in time t₁, so that a combined stream comprising methanol, hydrogen peroxide, water and propylene is obtained;
(iii-b) filtrating the combined stream obtained according to (ii-b) at a point in time t₂ through a filtration device, thereby obtaining a liquid mixture comprising methanol, hydrogen peroxide, water and propylene;
(iv-b) bringing the liquid mixture obtained according to (iii-b) in an epoxidation zone in contact with an epoxidation catalyst comprising a zeolitic material having a framework structure comprising Si, O, and Ti under epoxidation reaction conditions, thereby obtaining in the epoxidation zone a mixture comprising propylene oxide, methanol and water;
wherein the period of time between 1 and t₂ are at least 8 seconds.

According to this embodiment, the liquid mixture comprising methanol, hydrogen peroxide, water and propylene according to (iii-b) is further transferred to a reactor which comprises the epoxidation zone, the liquid mixture comprising methanol, hydrogen peroxide, water and propylene obtained in (iii-b) enters the reactor from any suitable direction (either side or top/bottom). In a preferred constellation wherein a horizontally arranged reactor is used, the liquid mixture obtained in (iii-b) enters the reactor from the side. Preferably, a constellation with a vertically arranged reactor is used, wherein the liquid mixture obtained in (iv-b) enters the reactor from the bottom. More preferably, a constellation with a vertically arranged reactor is used, wherein the liquid mixture obtained in (iv-b) enters the reactor from the bottom and flow direction in the reactor and through the epoxidation zone is from bottom to top.

Details regarding the residence time, methanol, water and hydrogen peroxide, the ratios in the combined stream prior to filtration, the ratios in liquid mixture and the filtration device, as well as the temperature ranges of the feed streams, combined streams and liquid mixtures, are as disclosed above in the section related to the first aspect.

### Epoxidation Catalyst

According to a preferred embodiment of the method for the preparation of propylene, the zeolitic material comprises Ti in an amount in the range of from 0.2 to 5 weight-%, preferably in the range of from 0.5 to 4 weight-%, more preferably in the range of from 1.0 to 3 weight-%, more preferably in the range of from 1.2 to 2.5 weight-%, more preferably in the range of from 1.4 to 2.2 weight-%, calculated as elemental Ti and based on the total weight of the zeolitic material.

According to a preferred embodiment of the method for the preparation of propylene, the zeolitic material having a framework structure comprising Si, O and Ti comprised in the epoxidation catalyst is a titanium zeolite having ABW, ACO, AEI, AEL, AEN, AET, AFG, AFI, AFN, AFO, AFR, AFS, AFT, AFX, AFY, AHT, ANA, APC, APD, AST, ASV, ATN, ATO, ATS, ATT, ATV, AWO, AWW, BCT, BEA, BEC, BIK, BOG, BPH, BRE, CAN, CAS, CDO, CFI, CGF, CGS, CHA, CHI, CLO, CON, CZP, DAC, DDR, DFO, DFT, DOH, DON, EAB, EDI, EMT, EPI, ERI, ESV, ETR, EUO, FAU, FER, FRA, GIS, GIU, GME, GON, GOO, HEU, IFR, ISV, ITE, ITH, ITQ, ITW, IWR, IWW, JBW, KFI, LAU, LEV, LIO, LOS, LOV, LTA, LTL, LTN, MAR, MAZ, MCM-22(S), MCM-36, MCM-56, MEI, MEL, MEP, MER, MIT-1, MMFI, MFS, MON, MOR, MSE, MSO, MTF, MTN, MTT, MTW, MWW, NAB, NAT, NEES, NON, NPO, OBW, OFF, OSI, OSO, PAR, PAU, PHI, PON, RHO, RON, RRO, RSN, RTE, RTH, RUT, RWR, RWY, SAO, SAS, SAT, SAV, SBE, SBS, SBT, SFE, SFF, SFG, SFH, SFN SFO, SGT, SOD, SSY, STF, STI, STT, TER, THO, TON, TSC, UEI, UFI, UOZ, USI, UTL, VET, VFI, VNI, VSV, WEI, WEN, YUG, ZON, SVR, SVY framework type or a mixed structure of two or more of these framework types; more preferably the zeolitic material having a framework structure comprising Si, O and Ti is a titanium zeolite having an MFI framework type, an MEL framework type, an MWW framework type, an MCM-22(S) framework type, an MCM-56 framework type, an IEZ-MWW framework type, an MCM-36 framework type, an ITQ framework type, a BEA framework type, a MOR framework type, or a mixed structure of two or more of these framework types; more preferably an MFI framework type, or an MWW framework type; more preferably the zeolitic material having a framework structure comprising Si, O and Ti has framework type MFI; more preferably the zeolitic material having a framework structure comprising Si, O and Ti is a titanium silicalite-1 (TS-1).

According to a preferred embodiment of the method for the preparation of propylene, the epoxidation catalyst is in the form of a molding, more preferred in the form of an extrudate or a granule. According to a preferred embodiment of the method for the preparation of propylene, the epoxidation catalyst, preferably the molding, more preferred the extrudate or the granule is used in pellet form (catalyst pellets), wherein the pellets have a characteristic diameter in the range of from 1 to 3 mm. An extrudate has preferably a cylindrical form (cut round extrudate strands), a granule has preferably a spherical form, wherein both forms can be named “pellets”. The characteristic diameter of a pellet is the diameter in case of cylindrical pellet and also the diameter in the case of a spherical pellet, which is preferably made by granulation. “Spherical” comprises sphere and ellipsoid. “Diameter” means the characteristic size of a spherical pellet, which in case of a sphere is its diameter and in case of an ellipsoid is its shortest axis.

Preferably, the epoxidation catalyst, more preferably the molding, further comprises a binder. Preferably from 95 to 100 weight-%, more preferably from 98 to 100 weight-%, more preferably from 99 to 100 weight-%, more preferably from 99.5 to 100 weight-%, more preferably from 99.9 to 100 weight-% of the molding consist of the zeolitic material and the binder. Preferably from 95 to 100 weight-%, more preferably from 98 to 100 weight-%, more preferably from 99 to 100 weight-%, more preferably from 99.5 to 100 weight-%, more preferably from 99.9 to 100 weight-% of the binder comprised in the molding consist of Si and O.

According to a preferred embodiment of the method for the preparation of propylene, the epoxidation catalyst, preferably the molding, comprises the binder, calculated as SiO₂, in an amount in the range of from 2 to 90 weight-%, preferably in the range of from 5 to 70 weight-%, more preferably in the range of from 10 to 50 weight-%, more preferably in the range of from 15 to 30 weight-%, more preferably in the range of from 20 to 25 weight-%, based on the total weight of the epoxidation catalyst, preferably based on the total weight of the molding and/or wherein the epoxidation catalyst, preferably the molding, comprises the zeolitic material in an amount in the range of from 10 to 98 weight-%, preferably in the range of from 30 to 95 weight-%, more preferably in the in the range of from 50 to 90 weight-%, more preferably in the range of from 70 to 85 weight-%, more preferably in the range of from 75 to 80 weight-%, based on the total weight of the epoxidation catalyst, preferably based on the total weight of the molding.

### Additives/Buffer

According to a preferred embodiment of the method for the preparation of propylene, an additive is provided to the epoxidation zone, preferably an aqueous solution of an additive, so that the liquid mixture comprising organic solvent, hydrogen peroxide, water and propylene, which is brought in the epoxidation zone in contact with the epoxidation catalyst, additionally comprises an additive. The additive is preferably selected from the group consisting of potassium salt, ammonia, ammonium salt, etidronic acid, salt of etidronic acid and mixtures of two or more thereof, more preferably selected from the group consisting of potassium dihydrogen phosphate, dipotassium hydrogen phosphate, potassium formate, potassium acetate, potassium hydrogen carbonate, etidronic acid, ammonium dihydrogen phosphate, diammonium hydrogen phosphate, ammonia and mixtures of two or more thereof, preferably form the group consisting pf potassium dihydrogen phosphate, dipotassium hydrogen phosphate, etidronic acid, ammonia and mixtures of two or more thereof, wherein the additive more preferably comprises at least dipotassium hydrogen phosphate.

### Epoxidation Conditions

Generally, no specific restrictions exist regarding the conditions under which the contacting in the epoxidation zone with the epoxidation catalyst takes place provided that an efficient epoxidation of propylene takes place.

According to a preferred embodiment of the method for the preparation of propylene, the contacting under epoxidation reaction conditions in the epoxidation zone with the epoxidation catalyst is carried out at an absolute pressure in the epoxidation zone in the range of from 0.5 to 5.0 MPa, preferably in the range of from 1.5 to 3.0 MPa, more preferably in the range of from 1.8 to 2.8 MPa. The contacting under epoxidation reaction conditions in the epoxidation zone with the epoxidation catalyst is preferably carried out at a temperature in the epoxidation zone in the range of from 20 to 75° C., more preferably in the range of from 22 to 75° C., more preferably in the range of from 24 to 70° C., more preferably in the range of from 25 to 65° C. The temperature in the epoxidation zone in the context of this application is defined as the entrance temperature of the cooling medium to the mantle of the reactor. In case there is more than one entrance or even more than one reaction zone each with a separate entrance for the cooling medium, then the temperature in the reaction zone will be defined as the weight averaged temperature of all the cooling medium feeding streams.

Preferably, the epoxidation reaction conditions according to (v) comprise trickle-bed conditions or fixed-bed conditions, wherein fixed-bed conditions are more preferred. Preferably, these conditions are applied in a reactor wherein the catalyst is present in a fixed-bed. “Trickle-bed conditions” preferably mean that the reaction is preferably carried out at temperatures and pressures at which the reaction mixture is present partly in a liquid phase and partly in a gaseous phase, with the catalyst being present in a fixed bed. In embodiments with fixed-bed conditions, the reaction is preferably carried out at temperatures and pressures at which the reaction mixture is liquid and no gas phase is present in the epoxidation zone, wherein two or more liquid phases may exist, with the catalyst being present in a fixed bed.
(vi) bringing the liquid mixture obtained according to (iv) in an epoxidation zone in contact with an epoxidation catalyst comprising a zeolitic material having a framework structure comprising Si, O, and Ti under epoxidation reaction conditions, thereby obtaining in the epoxidation zone a mixture comprising propylene oxide, methanol and water;

Generally, the contacting of the liquid mixture provided in (v) in the epoxidation zone with the epoxidation catalyst can be carried out in any appropriate way. Thus, for example, it can be carried out in a batch reactor or in at least one semi-continuously operated reactor or in at least one continuously operated reactor. The continuous mode of operation is preferred, wherein preferably at least (v) is carried out continuously, wherein more preferably (i), (ii), (iii), (iv) and (v) are carried out continuously.

Preferably, the contacting of the liquid mixture with an epoxidation catalyst is carried out in at least one, preferably continuously operated, reactor such as a tube reactor or a tube bundle reactor which preferably contains at least one cooling jacket surrounding the at least one tube. A cooling medium flows through the cooling jacket. The nature of the cooling medium is not particular restricted as long as it is sufficient for adjusting the temperature in the epoxidation zone. For example, the cooling medium comprises water, wherein it may additionally comprise additives such as aliphatic C2 to C5 mono-alcohols, aliphatic C2 to C5 di-alcohols and mixtures of two or more thereof. Preferably, ≥90 weight-%, more preferred ≥95 weight-% of the cooling medium are water, based on the total weight of the cooling medium. The temperature of the cooling medium is the temperature of the cooling medium used for adjusting the temperature of the reaction mixture in epoxidation zone according to (v) wherein it is preferred that said temperature is adjusted by passing the cooling medium through a cooling jacket, wherein the temperature of the cooling medium is preferably the temperature of the cooling medium prior to adjusting the temperature of the reaction mixture, preferably the temperature of the cooling medium at the entrance of the cooling jacket.

### Epoxidation Zone

According to step (v) of the method for the preparation of propylene oxide, the liquid mixture is contacted in an epoxidation zone with an epoxidation catalyst comprising a zeolitic material having a framework structure comprising Si, O, and Ti, and the reaction mixture is subjected to epoxidation reaction conditions in the epoxidation zone, obtaining, in the epoxidation zone, a mixture comprising propylene oxide, methanol and water.

Generally, there are no specific restrictions regarding the design of the epoxidation zone provided that it is suitable for carrying out a, preferably continuous, epoxidation reaction. Preferably, the epoxidation zone according to (v) comprises one or more epoxidation subzone wherein a given epoxidation subzone preferably consist of one or more epoxidation reactors wherein, with regard to the design of the one or more epoxidation reactors, no specific restrictions exist provided that the reactors are suitable for carrying out a, preferably continuous, epoxidation reaction.

Preferably, the epoxidation zone according to (v) comprises a first epoxidation subzone consisting of one or more epoxidation reactors A. The term “first epoxidation subzone” as used in this context of the present invention relates to the epoxidation subzone into which the liquid mixture is passed, wherein the epoxidation zone of (v) may comprise further epoxidation subzones which are arranged downstream of the first epoxidation subzone. If the first epoxidation subzone consisting of two or more epoxidation reactors A, it is preferred that the two or more epoxidation reactors A are arranged in parallel. In this case, it is preferred that in (v), the liquid mixture is passed into at least one of the epoxidation reactors A. It is possible, for example, that, while the liquid mixture is passed into at least one of the epoxidation reactors A, at least one of the reactors A is taken out of operation, for example for maintenance purposes and/or for regenerating the catalyst comprised in the at least one of the reactors A. If the first epoxidation subzone comprises two or more epoxidation reactors A, the reactors in operation are operated essentially identically so that in every epoxidation reactor A in operation, a given epoxidation condition is in the same range in every reactor. For example, the temperature in the epoxidation zone is in the same range in every reactor.

The temperature of the cooling medium is the temperature of the cooling medium used for adjusting the temperature of the liquid mixture in the first epoxidation reaction subzone according to (v) wherein it is preferred that said temperature is adjusted by passing the cooling medium through a cooling jacket of the one or more epoxidation reactors A, wherein the temperature of the cooling medium is preferably the temperature of the cooling medium prior to adjusting the temperature of the reaction mixture, preferably the temperature of the cooling medium at the entrance of the cooling jacket of the one or more epoxidation reactors A. If the first epoxidation subzone comprises two or more epoxidation reactors A, the temperature of the cooling medium relates a given reactor A in operation of the first epoxidation subzone.

According to a first preferred embodiment of the method, the epoxidation zone according to (v) consists of the first epoxidation subzone. According to a second preferred embodiment of the method, the epoxidation zone according to (v) additionally comprises a second epoxidation subzone consisting of one or more epoxidation reactors B wherein, if the second epoxidation subzone comprises two or more epoxidation reactors B, the two or more epoxidation reactors B are arranged in parallel, wherein the second epoxidation subzone is arranged downstream of the first epoxidation subzone. In this case, it is preferred that the effluent stream obtained from the first epoxidation subzone, optionally after a suitable intermediate treatment, is passed into at least one of the epoxidation reactors B. It is possible, for example, that, while the effluent stream obtained from the first epoxidation subzone, optionally after a suitable intermediate treatment, is passed into at least one of the epoxidation reactors B, at least one of the reactors B is taken out of operation, for example for maintenance purposes and/or for regenerating the catalyst comprised in the at least one of the reactors B. If the second epoxidation subzone comprises two or more epoxidation reactors B, the reactors in operation are operated essentially identically so that in every epoxidation reactor B in operation, a given epoxidation condition is in the same range in every reactor. Generally, it is conceivable that in addition to the first epoxidation subzone and the second epoxidation subzone, the epoxidation zone according to (v) comprises at least one further epoxidation subzone arranged downstream of the second epoxidation subzone. Preferably, according to the second preferred embodiment of the present invention, the epoxidation zone according to (v) consists of the first epoxidation subzone and the second epoxidation subzone.

Preferably, the temperature of the liquid mixture in the second epoxidation reaction subzone is not adjusted by passing a cooling medium through a cooling jacket of the one or more epoxidation reactors B. More preferably, the second epoxidation subzone is an essentially adiabatic epoxidation subzone. More preferably, the second epoxidation subzone is an adiabatic epoxidation subzone.

Subsequent to steps (i), (ii), (iii), (iv) and (v) the process for the preparation of propylene oxide may comprise
(vi) separating propylene oxide from the mixture comprising propylene oxide, methanol and water obtained in (v), optionally after having separated propylene, obtaining a stream S1 comprising propylene oxide and a stream S2 comprising water and methanol.

Preferably, (vi) comprises
(vi-1) separating propylene from the mixture comprising propylene oxide, methanol and water obtained in (v) and preferably recycling at least a portion of the separated propylene, optionally after work-up, to the epoxidation zone according to (v), obtaining a stream S1a comprising propylene oxide, water, and methanol and being depleted of propylene compared to the mixture obtained in (v);
(vi-2) separating propylene oxide from the stream S1a obtained according to (vi-1), obtaining a stream S1b comprising propylene oxide and being depleted of water and methanol compared to the mixture obtained in (v) and stream S1a respectively, and a stream S2 comprising water and methanol and being depleted of propylene oxide compared to the mixture and S1a respectively.

Subsequent to steps (i), (ii), (iii), (iv), (v) and (vi) the method for the preparation of propylene oxide may comprise
(vii) separating methanol from stream S2 obtained according to (vi) or (vi-2) by distillation, obtaining a stream S3 enriched in methanol compared to stream S2 and preferably recycling at least a portion of S3, optionally after work-up, to the epoxidation zone according to (v).

It goes without saying that a wording “subsequent to steps (i), (ii), (iii), (iv) and (v)” means that step (vi) is carried out after, preferably directly after, step (v). “Directly after” means that no intermediate steps are carried out between (v) and (vi). It is understood that this also means that the complete sequence of steps is preferably (i), (ii), (iii), (iv), (v) and (vi) in that order, wherein preferably no intermediate step is carried out in between. The same applies for step vii).

### 4^{th} Aspect—Propylene Oxide

A fourth aspect of the present invention is related to propylene oxide, obtained or obtainable from the method according to the third aspect.

The present invention is further illustrated by the following embodiments and combinations of embodiments as indicated by the respective dependencies and back-references. In particular, it is noted that in each instance where a range of embodiments is mentioned, for example in the context of a term such as “The process of any one of embodiments 1 to 4”, every embodiment in this range is meant to be explicitly disclosed for the skilled person, i.e. the wording of this term is to be understood by the skilled person as being synonymous to “The process of any one of embodiments 1, 2, 3, and 4”. Further, it is explicitly noted that the following set of embodiments is not the set of claims determining the extent of protection, but represents a suitably structured part of the description directed to general and preferred aspects of the present invention.
1. A process for the preparation of a liquid mixture comprising methanol, water and hydrogen peroxide, the process comprising
   (iv) providing a feed stream comprising methanol, and a feed stream comprising an aqueous hydrogen peroxide solution;
   (v) combining the feed stream comprising the methanol and the feed stream comprising the aqueous hydrogen peroxide solution provided according to (i) at a point in time t₁, so that a combined stream comprising methanol, hydrogen peroxide and water is obtained;
   (vi) filtrating the combined stream obtained according to (ii) at a point in time t₂, through a filtration device, thereby obtaining a liquid mixture comprising methanol, hydrogen peroxide and water;
   wherein the period of time between 1 and t₂ is at least 8 seconds.
2. The process for the preparation of a liquid mixture comprising methanol, water and hydrogen peroxide according to embodiment 1, comprising
   (v) adding to the feed stream comprising methanol a further feed stream comprising propylene before (ii) or adding to the liquid mixture obtained in (iii) a further feed stream comprising propylene,
   thereby obtaining a liquid mixture comprising methanol, hydrogen peroxide, water and propylene.
3. The process for the preparation of a liquid mixture comprising methanol, water and hydrogen peroxide according to embodiment 1 or 2, comprising
   (i) providing a feed stream comprising methanol and a feed stream comprising an aqueous hydrogen peroxide solution;
   (ii-a) combining the feed stream comprising the methanol and the feed stream comprising the aqueous hydrogen peroxide solution provided according to (i) at a point in time t₁, so that a combined stream comprising methanol, hydrogen peroxide and water is obtained;
   (iii-a) filtrating the combined stream obtained according to (ii-a) at a point in time t₂, through a filtration device, thereby obtaining a liquid mixture comprising methanol, hydrogen peroxide and water;
   (iv-a) adding to the liquid mixture obtained in (iii-a) a further feed stream comprising propylene, thereby obtaining a liquid mixture comprising methanol, hydrogen peroxide, water and propylene;
   wherein the period of time between t₁ and t₂ are at least 8 seconds.
4. The process for the preparation of a liquid mixture comprising methanol, water and hydrogen peroxide according to embodiment 1, comprising
   (i) providing a feed stream comprising methanol and a feed stream comprising an aqueous hydrogen peroxide solution;
   (ii-b) combining the feed stream comprising the methanol with a further feed stream comprising propylene thereby obtaining a stream comprising methanol and propylene;
   (iii-b) combining the stream comprising methanol and propylene obtained in (ii-b) and the feed stream comprising the aqueous hydrogen peroxide solution at a point in time t₁, so that a combined stream comprising methanol, hydrogen peroxide, water and propylene is obtained;
   (iv-b) filtrating the combined stream obtained according to (iii-b) at a point in time t₂ through a filtration device, thereby obtaining a liquid mixture comprising methanol, hydrogen peroxide, water and propylene;
   wherein the period of time between t₁ and t₂ are at least 8 seconds.
5. The process for the preparation of a liquid mixture comprising methanol, water and hydrogen peroxide according to any one of embodiments 1 to 4, wherein the period of time between t₁ and t₂ is a period in the range of from 8 seconds to 5 hours, more preferably in the range of from 8 seconds to 4 hours, more preferably in the range of from 8 seconds to 3 hours, more preferably in the range of from 8 seconds to 2 hours, more preferably in the range of from 8 seconds to 1 hour.
6. The process for the preparation of a liquid mixture comprising methanol, water and hydrogen peroxide according to any one of embodiments 1 to 5, wherein the hydrogen peroxide is provided as aqueous hydrogen peroxide solution, which has a total organic carbon content (TOC) in the range of from 100 to 800 mg per kg hydrogen peroxide comprised in the aqueous hydrogen peroxide solution, preferably in the range of from 120 to 750 mg per kg hydrogen peroxide comprised in the aqueous hydrogen peroxide solution, more preferably in the range of from 150 to 700 mg per kg hydrogen peroxide comprised in the aqueous hydrogen peroxide solution, determined according to DIN EN 1484 (April 2019).
7. The process for the preparation of a liquid mixture comprising methanol, water and hydrogen peroxide according to any one of embodiments 1 to 6, wherein the aqueous hydrogen peroxide solution has a pH in the range of from 0 to 3.0, preferably in the range of from 0.1 to 2.5, more preferably in the range of from 0.5 to 2.3, determined with a pH sensitive glass electrode according to CEFIC PEROXYGENS H2O2 AM-7160 standard (2003).
8. The process for the preparation of a liquid mixture comprising methanol, water and hydrogen peroxide according to any one of embodiments 1 to 7, wherein the aqueous hydrogen peroxide solution comprises from 20 to 85 weight-%, preferably from 30 to 75 weight-%, more preferably from 40 to 70 weight-% of hydrogen peroxide, relative to the total weight of the aqueous hydrogen peroxide solution.
9. The process for the preparation of a liquid mixture comprising methanol, water and hydrogen peroxide according to any one of embodiments 1 to 8, wherein the hydrogen peroxide, preferably the aqueous hydrogen peroxide solution, is obtained or obtainable from an anthraquinone process.
10. The process for the preparation of a liquid mixture comprising methanol, water and hydrogen peroxide according to any one of embodiments 1 to 9, wherein the aqueous hydrogen peroxide solution contains in the range of from 0.1 to 10 mg of non-alkali metal cations per kg of hydrogen peroxide, preferably in the range of from 0.25 to 5 mg of non-alkali metal cations per kg of hydrogen peroxide, wherein the non-alkali metal cations are preferably selected from the group consisting of cations of Si, Fe, Ni, Mn, Al, Cr, Pd, Ca, Mg and mixtures of two or more of these metal cations.
11. The process for the preparation of a liquid mixture comprising methanol, water and hydrogen peroxide according to any one of embodiments 1 to 10, wherein the aqueous hydrogen peroxide solution is stabilized with a stabilizer selected from the group consisting of phosphoric acid, pyrophosphoric acid, nitric acid, dialkali hydrogenphosphate, alkali dihydrogen phosphate, dialkali pyrophosphate, tetraalkali pyrophosphate, ammonium nitrate, alkali nitrate and mixtures of two or more of these stabilizers, wherein the alkali metal is preferably sodium or potassium, more preferably sodium.
12. The process for the preparation of a liquid mixture comprising methanol, water and hydrogen peroxide according to embodiment 11, wherein the aqueous hydrogen peroxide solution comprises in the range of from 0 to 0.010 weight-% sodium cations (0 to 100 weight-ppm), and in the range of from 0.001 to 0.050 weight-% (10 to 500 weight-ppm), preferably in the range of from 0.005 to 0.025 weight-% (50 to 250 weight-ppm) phosphorus, calculated as phosphate (PO₄³⁻), each based on the total weight of the aqueous hydrogen peroxide solution.
13. The process for the preparation of a liquid mixture comprising methanol, water and hydrogen peroxide according to any one of embodiments 1 to 12, wherein the weight ratio of methanol:hydrogen peroxide (w/w) in the combined stream obtained in (ii), (ii-a) or (ii-b) is in the range of from 15:1 to 5:1, preferably in the range of from 12:1 to 6:1, more preferably in the range of from 12:1 to 9:1 or in the range of from 8:1 to 6:1.
14. The process for the preparation of a liquid mixture comprising methanol, water and hydrogen peroxide according to any one of embodiments 2 to 13, wherein the weight ratio of propylene:hydrogen peroxide (w/w) in the liquid mixture, which is obtained in (iv), (iv-a) or (iii-b), is in the range of from 1:1 to 5:1, preferably in the range of from 1:1 to 2:1 or in the range of from 3:1 to 5:1.
15. The process for the preparation of a liquid mixture comprising methanol, water and hydrogen peroxide according to any one of embodiments 2 to 14, wherein the weight ratio of methanol:hydrogen peroxide (w/w) in the liquid mixture, which is obtained in (iv), (iv-a) or (iii-b), is in the range of from 15:1 to 5:1, preferably in the range of from 12:1 to 6:1, more preferably in the range of from 12:1 to 9:1 or in the range of from 8:1 to 6:1.
16. The process for the preparation of a liquid mixture comprising methanol, water and hydrogen peroxide according to any one of embodiments 2 to 15, wherein the weight ratio of methanol:propylene (w/w) in the liquid mixture, which is obtained in (iv), (iv-a) or (iii-b), is in the range of from 10:1 to 1:0.1, preferably in the range of from 9:1 to 1:1, more preferably in the range of from 9:1 to 7:1 or in the range of from 1.5:1 to 1:1.
17. The process for the preparation of a liquid mixture comprising methanol, water and hydrogen peroxide according to any one of embodiments 1 to 16, wherein the filtration device according to (iii), (iii-a) and/or (iii-b) is a filter, preferably a filter selected from the group consisting of pleated filter, CUNO type filter and mixed forms of these filter types.
18. The process for the preparation of a liquid mixture comprising methanol, water and hydrogen peroxide according to embodiments 17, wherein the filter is operated with a specific loading, which is the quotient of the volume flow rate per area in the range of from 0.05×10⁻⁴ to 500×10⁻⁴ m/s, preferably in the range of from 0.5×10⁻⁴ to 50×10⁻⁴ m/s, more preferably in the range of from 1×10⁻⁴ to 10×10⁻⁴ m/s.
19. The process for the preparation of a liquid mixture comprising methanol, water and hydrogen peroxide according to embodiment 16 or 17, wherein the filter is a pleated filter, preferably a pleated filter wherein the pleats are at least partially laid over each other.
20. The process for the preparation of a liquid mixture comprising methanol, water and hydrogen peroxide according to embodiment 19, wherein the pleated filter is surrounded by external supporting elements.
21. The process for the preparation of a liquid mixture comprising methanol, water and hydrogen peroxide according to any one of embodiments 17 to 20, wherein the filter material comprises, preferably consists of, polypropylene or polyethylene, preferably polypropylene (PP).
22. Use of a liquid mixture obtained or obtainable from the process of any of the embodiments 1 to 21 for the preparation of propylene oxide.
23. A method for the preparation of propylene oxide comprising
   (i) providing a feed stream comprising methanol, a feed stream comprising an aqueous hydrogen peroxide solution and a feed stream comprising propylene;
   (ii) combining the feed stream comprising the methanol and the feed stream comprising the aqueous hydrogen peroxide solution provided according to (i) at a point in time t₁, so that a combined stream comprising methanol, hydrogen peroxide and water is obtained;
   (iii) filtrating the combined stream obtained according to (ii) at a point in time t₂, through a filtration device, thereby obtaining a liquid mixture comprising methanol, hydrogen peroxide and water;
   (iv) combining the feed stream comprising propylene either with the combined stream obtained in (ii) or with the liquid mixture obtained in (iii), thereby obtaining a liquid mixture comprising methanol, hydrogen peroxide, water and propylene;
   (v) bringing the liquid mixture obtained according to (iv) in an epoxidation zone in contact with an epoxidation catalyst comprising a zeolitic material having a framework structure comprising Si, O, and Ti under epoxidation reaction conditions, thereby obtaining in the epoxidation zone a mixture comprising propylene oxide, methanol and water;
   wherein the period of time between t₁ and t₂ are at least 8 seconds.
24. The method for the preparation of propylene according to embodiment 23, wherein the zeolitic material comprises Ti in an amount in the range of from 0.2 to 5 weight-%, preferably in the range of from 0.5 to 4 weight-%, more preferably in the range of from 1.0 to 3 weight-%, more preferably in the range of from 1.2 to 2.5 weight-%, more preferably in the range of from 1.4 to 2.2 weight-%, calculated as elemental Ti and based on the total weight of the zeolitic material.
25. The method for the preparation of propylene according to embodiment 23 or 24, wherein the zeolitic material having a framework structure comprising Si, O and Ti comprised in the epoxidation catalyst is a titanium zeolite having ABW, ACO, AEI, AEL, AEN, AET, AFG, AFI, AFN, AFO, AFR, AFS, AFT, AFX, AFY, AHT, ANA, APC, APD, AST, ASV, ATN, ATO, ATS, ATT, ATV, AWO, AWW, BCT, BEA, BEC, BIK, BOG, BPH, BRE, CAN, CAS, CDO, CFI, CGF, CGS, CHA, CHI, CLO, CON, CZP, DAC, DDR, DFO, DFT, DOH, DON, EAB, EDI, EMT, EPI, ERI, ESV, ETR, EUO, FAU, FER, FRA, GIS, GIU, GME, GON, GOO, HEU, IFR, ISV, ITE, ITH, ITQ, ITW, IWR, IWW, JBW, KFI, LAU, LEV, LIO, LOS, LOV, LTA, LTL, LTN, MAR, MAZ, MCM-22(S), MCM-36, MCM-56, MEI, MEL, MEP, MER, MIT-1, MMFI, MFS, MON, MOR, MSE, MSO, MTF, MTN, MTT, MTW, MWW, NAB, NAT, NEES, NON, NPO, OBW, OFF, OSI, OSO, PAR, PAU, PHI, PON, RHO, RON, RRO, RSN, RTE, RTH, RUT, RWR, RWY, SAO, SAS, SAT, SAV, SBE, SBS, SBT, SFE, SFF, SFG, SFH, SFN SFO, SGT, SOD, SSY, STF, STI, STT, TER, THO, TON, TSC, UEI, UFI, UOZ, USI, UTL, VET, VFI, VNI, VSV, WEI, WEN, YUG, ZON, SVR, SVY framework type or a mixed structure of two or more of these framework types; more preferably the zeolitic material having a framework structure comprising Si, O and Ti is a titanium zeolite having an MFI framework type, an MEL framework type, an MWW framework type, an MCM-22(S) framework type, an MCM-56 framework type, an IEZ-MWW framework type, an MCM-36 framework type, an ITQ framework type, a BEA framework type, a MOR framework type, or a mixed structure of two or more of these framework types; more preferably an MFI framework type, or an MWW framework type; more preferably the zeolitic material having a framework structure comprising Si, O and Ti has framework type MFI; more preferably the zeolitic material having a framework structure comprising Si, O and Ti is a titanium silicalite-1 (TS-1).
26. The method for the preparation of propylene according to any one of embodiments 23 to 25, wherein the epoxidation catalyst is in the form of a molding, more preferred in the form of an extrudate or a granule.
27. The method for the preparation of propylene according to any one of embodiments 23 to 26, wherein the epoxidation catalyst, preferably the molding, more preferred the extrudate or the granule is used in pellet form (catalyst pellets), wherein the pellets have a characteristic diameter in the range of from 1 to 3 mm.
28. The method for the preparation of propylene according to any one of embodiments 23 to 27, wherein the epoxidation catalyst, preferably the molding, further comprises a binder.
29. The method for the preparation of propylene according to any one of embodiments 23 to 28, wherein from 95 to 100 weight-%, preferably from 98 to 100 weight-%, more preferably from 99 to 100 weight-%, more preferably from 99.5 to 100 weight-%, more preferably from 99.9 to 100 weight-% of the molding consist of the zeolitic material and the binder.
30. The method for the preparation of propylene according to any one of embodiments 23 to 29, wherein from 95 to 100 weight-%, preferably from 98 to 100 weight-%, more preferably from 99 to 100 weight-%, more preferably from 99.5 to 100 weight-%, more preferably from 99.9 to 100 weight-% of the binder comprised in the molding consist of Si and O.
31. The method for the preparation of propylene according to any one of embodiments 23 to 30, wherein the epoxidation catalyst, preferably the molding, comprises the binder, calculated as SiO₂, in an amount in the range of from 2 to 90 weight-%, preferably in the range of from 5 to 70 weight-%, more preferably in the range of from 10 to 50 weight-%, more preferably in the range of from 15 to 30 weight-%, more preferably in the range of from 20 to 25 weight-%, based on the total weight of the epoxidation catalyst, preferably based on the total weight of the molding and/or wherein the epoxidation catalyst, preferably the molding, comprises the zeolitic material in an amount in the range of from 10 to 98 weight-%, preferably in the range of from 30 to 95 weight-%, more preferably in the in the range of from 50 to 90 weight-%, more preferably in the range of from 70 to 85 weight-%, more preferably in the range of from 75 to 80 weight-%, based on the total weight of the epoxidation catalyst, preferably based on the total weight of the molding.
32. The method for the preparation of propylene according to any one of embodiments 23 to 31, wherein an additive is provided to the epoxidation zone, preferably an aqueous solution of an additive, so that the liquid mixture comprising organic solvent, hydrogen peroxide, water and propylene, which is brought in the epoxidation zone in contact with the epoxidation catalyst, additionally comprises an additive.
33. The method for the preparation of propylene according to embodiment 32, wherein the additive is selected from the group consisting of potassium salt, ammonia, ammonium salt, etidronic acid, salt of etidronic acid and mixtures of two or more thereof, preferably selected from the group consisting of potassium dihydrogen phosphate, dipotassium hydrogen phosphate, potassium formate, potassium acetate, potassium hydrogen carbonate, etidronic acid, ammonium dihydrogen phosphate, diammonium hydrogen phosphate, ammonia and mixtures of two or more thereof, preferably form the group consisting pf potassium dihydrogen phosphate, dipotassium hydrogen phosphate, etidronic acid, ammonia and mixtures of two or more thereof, wherein the additive more preferably comprises at least dipotassium hydrogen phosphate.
34. The method for the preparation of propylene according to any one of embodiments 23 to 33, wherein the contacting under epoxidation reaction conditions in the epoxidation zone with the epoxidation catalyst is carried out at an absolute pressure in the epoxidation zone in the range of from 0.5 to 5.0 MPa, preferably in the range of from 1.5 to 3.0 MPa, more preferably in the range of from 1.8 to 2.8 MPa.
35. The method for the preparation of propylene according to any one of embodiments 23 to 34, wherein the contacting under epoxidation reaction conditions in the epoxidation zone with the epoxidation catalyst is carried out at a temperature in the epoxidation zone in the range of from 20 to 75° C., preferably in the range of from 22 to 75° C., more preferably in the range of from 24 to 70° C., more preferably in the range of from 25 to 65° C.
36. The method for the preparation of propylene according to any one of embodiments 23 to 35, wherein the epoxidation reaction conditions comprise trickle bed conditions.
37. The method for the preparation of propylene according to any one of embodiments 23 to 36, wherein the epoxidation reaction conditions comprise fixed bed conditions.
38. Propylene oxide, obtained or obtainable from the method according to any one of embodiments 23 to 37.

The present invention is further illustrated by the following examples.

### EXAMPLES

### Reference Example 1: Experimental Set-Up

A mini-plant with a reaction tube and a main feed line towards the reaction tube was used in all experiments.

The main feed line to the reaction tube was a tube (feed tube) having at least two separate T-junctions T1 and T2 located at separate positions along the feed tube, wherein each T-junction could be used as feeding point for a stream which should be fed to the main feed line, and wherein T1 was positioned most far away from the entrance to the reaction tube, and T2 was the closest T-junction with respect to the entrance to the reaction tube. A filter was installed in the feed tube after the T-junction T2 but before entrance into the reaction tube, wherein a stainless-steel metal sinter filter with a nominal rating of 2 μm was used (Swagelok SS-2F-K4-2, the filter element is made of sintered stainless steel SS316 and has a filter surface of 350 mm²). A new filter was used for each (comparative) experiment. The filter was operated with a specific loading as indicated in the Comparative Examples 1 and 2 and the Example 1.

Two pressure transducers were used, one just upstream of the second T-junction T2 and one just downstream of the filter, to measure the pressure difference across the filter (deltaP). Further downstream of the filter, a pressure control valve maintained the pressure downstream from the filter in the feed tube. The expressions “upstream” and “downstream” are meant in relation to the flow of any feed stream in the feed tube towards the reaction tube.

The available volume between the second T-junction T2 and the filter could be varied by changing the length of the feed tube section connecting the T-junction T2 to the filter.

### Comparative Example 1: Experiment with Methanol Only

Methanol was fed to the main feed line at a rate of 370 g/h using a high-pressure membrane feed pump using an experimental setup according to Reference Example 1. The specific filter loading was 3.7×10−4 m/s. DeltaP was determined according to Reference Example 1: deltaP was in average 220 mbar and remained constant over a period of 3 hours, i.e. for a period of time of 3 hours, no pressure increase was observed.

### Comparative Example 2: Experiment with Methanol and Propylene Only

Methanol was fed to the main feed line at a rate of 370 g/h using a high-pressure membrane feed pump using an experimental setup according to Reference Example 1. Further, liquid propylene was fed to the first T-junction T1 at a rate of 54 g/g, using another high-pressure membrane feed pump. The specific filter loading of was now 4.4×10⁻⁴ m/s. deltaP was determined according to Reference Example 1: deltaP was in average 220 mbar and remained constant over a period of 3 hours, i.e. for a period of time of 3 hours, no pressure increase was observed.

### Example 1: Experiment with Methanol, Propylene and H₂O₂

Methanol was fed to the main feed line at a rate of 370 g/h using a high-pressure membrane feed pump using an experimental setup according to Reference Example 1. Liquid propylene was fed to the first T-junction T1 at a rate of 54 g/h, using another high-pressure membrane feed pump. Only methanol and propylene were fed for 3 hours. During this period, deltaP remained essentially constant at 200 mbar.

After these three hours, feeding of aqueous hydrogen peroxide solution via T2 was started in that aqueous hydrogen peroxide solution was fed to the section T-junction T2 at a rate of 98 g/h, using another high-pressure membrane feed pump. The aqueous hydrogen peroxide solution used was obtained from a plant using the antraquinone process and had a concentration of 39 weight-% and contained 97 weight-ppm of total phosphates (expressed as PO₄), each based on the total weight of the aqueous hydrogen peroxide solution. Feeding of methanol, propylene, hydrogen peroxide and water was continued for 4 hours. The specific filter loading was 5.24×10⁻⁴ m/s.

The available volume between the second T-junction T2 and the filter could be varied by changing the length of the feed tube section connecting the T-junction T2 to the filter, thus allowing to vary the residence time of the feed stream methanol, propylene, hydrogen peroxide and water after the second T-junction T2 before it reaches the filter. Due to the inherent volume of the second T-junction T2 and of the filter, the minimum residence time achievable with the setup used was 1.1 s.

The “residence time” was defined as the time span between point in time t₁, when the aqueous hydrogen peroxide solution was added at T-junction T2 and the point in time t₂, when the combined stream reached the filter (t₂−t₁).

DeltaP was determined according to Reference Example 1, wherein pressure readings were taken every 10 min. During the four hours of feeding methanol, propylene, hydrogen peroxide and water, deltaP increased in an approximately linear fashion and an average pressure increase rate versus time was calculated by using an ordinary linear least squares method. Separate experiments were carried out for residence times 1.1 seconds, 3.8 seconds, 6 seconds, 8 seconds, 20 seconds and 25 seconds. For every residence time, the experiment was performed twice to ensure reproducibility. The results for the average pressure increase rate are shown in the table 1 below and in FIG. 1. As can be seen from the table and the graphical representation in FIG. 1, a minimum residence time of 8 seconds was required until the solids formed and could be retained by the filter.

TABLE 1
Results for the average pressure increase rate

| Number of | Residence time between second T- | Rate of increase of |
|---|---|---|
| experiment | junction T2 and filter [seconds] | deltaP [bar/h] |
| 1 | 1.1 | 0.13 |
| 2 | 3.8 | 0.11 |
| 3 | 6.0 | 0.08 |
| 4 | 8.0 | 0.75 |
| 5 | 20.0 | 0.82 |
| 6 | 25.0 | 0.92 |

### Comparative Examples 3, 4 and Examples 2, 3: Decomposition of Hydrogen Peroxide

A 10:1 (w/w) mixture of methanol and an aqueous hydrogen peroxide solution with 40 weight-% hydrogen peroxide was analyzed by differential scanning calorimetry (DSC) in a glass jar (closed high pressure cell made of glass) in the absence (Comparative Example 3) or presence (Examples 2) of precipitate. The precipitate came from Example 1 and had previously been taken from the filter of the mini-plant described in Reference Example 1 and used in Example 1. DSC measurement and data collection was carried out in accordance with DIN 51007(2019-04) and DIN 51005 (2021-08) on a DSC device from Mettler Toledo. The sample used for Comparative example 3 had 11.6 mg total weight. The sample used for Example 2 contained 1% by weight of precipitate wherein the total weight was also 11.6 mg (100% by weight). Both samples were inertized with nitrogen. The temperature range of from 0 to 500° C. was scanned with a scan rate of 2.5 K/min. The analysis was made with the STARe Software package of Mettler Toledo, Version 16.30. The results are listed in Table 2 below.

TABLE 2
Decomposition of hydrogen peroxide in the presence or absence of
precipitate for a 10:1 (w/w) mixture of methanol and an aqueous
hydrogen peroxide solution with 40 weight-% hydrogen peroxide

| | Onset temperature [° C.] | Peak [° C.] |
|---|---|---|
| Comparative Example 3 | 113 | 156 |
| Example 2 | 68 | 120 |

It was found that in the presence of the precipitate, the onset temperature, i.e. the temperature, at which decomposition of hydrogen peroxide started, was reduced by 45° C. (from 113° C. to 68° C.), which showed that the precipitate functioned as a catalyst for the decomposition of hydrogen peroxide.

The Example and Comparative Example were repeated with 9.56 mg of a mixture comprising 90% by weight methanol and 10% by weight of an aqueous hydrogen peroxide solution with 40 weight-% hydrogen peroxide and 1.36 mg propylene, corresponding to an overall mixture comprising 78.8 weight-% methanol, 3.5 weight-% hydrogen peroxide, 5.2 weight-% water and 12.4% by weight propylene in case of absence of precipitate (Comparative Example 4) and, in case of presence of precipitate, with 15.87 mg of a mixture comprising 90% by weight methanol and 10% by weight of an aqueous hydrogen peroxide solution with 40 weight-% hydrogen peroxide, 1.81 mg propylene, corresponding to an overall mixture comprising 80.8 weight-% methanol, 3.6 weight-% hydrogen peroxide, 5.4 weight-% water and 10.1% by weight propylene, to which 0.17 mg precipitate were added (Example 3). Both experiments were conducted in a glass jar (closed high pressure cell made of glass). The overall mixtures were prepared in that methanol and aqueous hydrogen peroxide solution were mixed and filled into the glass jar. For Example 3, the precipitate was then added to the mixture. For Comparative Example 4, no precipitate was added. Subsequently, the cell was flushed with propylene and after flushing propylene was added to a final pressure of 4.5 bar at 20° C. Afterwards the glass jars were closed. T. The DSCs were recorded and analyzed as described above with respect to Comparative Example 3, Example 2. The results are listed below in Table 3.

TABLE 3
Decomposition of hydrogen peroxide in the presence
or absence of precipitate for a 9:1 (w/w) mixture
of methanol and an aqueous hydrogen peroxide solution
with 40 weight-% hydrogen peroxide with propylene

| | Onset temperature [° C.] | Peak [° C.] |
|---|---|---|
| Comparative Example 4 | 105 | 167 |
| Example 3 | 60 | 123 |

It was found that in the presence of the precipitate, the onset temperature was reduced by 45° C., which showed that the precipitate functioned as a catalyst for the decomposition of hydrogen peroxide.

### SUMMARY

Mixing aqueous hydrogen peroxide, especially aqueous hydrogen peroxide derived from an anthraquinone process, with an organic solvent such as methanol normally results in precipitate formation. In case such precipitate form on an epoxidation catalyst, the performance of the epoxidation catalyst and thus of the epoxidation reaction is substantially impaired. Thus, it is desirable to hinder such precipitate from forming in the reactor and also, if formed before entering the reactor, also hindering such precipitate from entering the reactor, which is normally done by use of filters.

When mixing aqueous hydrogen peroxide with an organic solvent such as methanol, it was shown that the precipitate formation required a certain amount of time after the aqueous hydrogen peroxide came in contact with the methanol. It could be shown that the residence time of a combined stream comprising methanol and aqueous hydrogen peroxide solution before coming in contact with a filter had to be at least 8 seconds. If the residence time was less than 8 seconds, the precipitate had not enough time to form in the feed tube before the filter but moreover formed after the filter, thus impairing the catalyst's performance and, consequently, the performance of the epoxidation reaction.

Furthermore, it was shown that an upper limit of the residence time was also essential in order to avoid precipitate formation in the feed line(s), since precipitate in the feed lines-aside from other detrimental effects-functioned as a catalyst for the decomposition of hydrogen peroxide.

### SHORT DESCRIPTION OF THE FIGURE

FIG. 1 shows the rate of increase of deltaP indicated in bar per hour on the y axis versus the residence time indicated in seconds on the x axis according to Example 1.

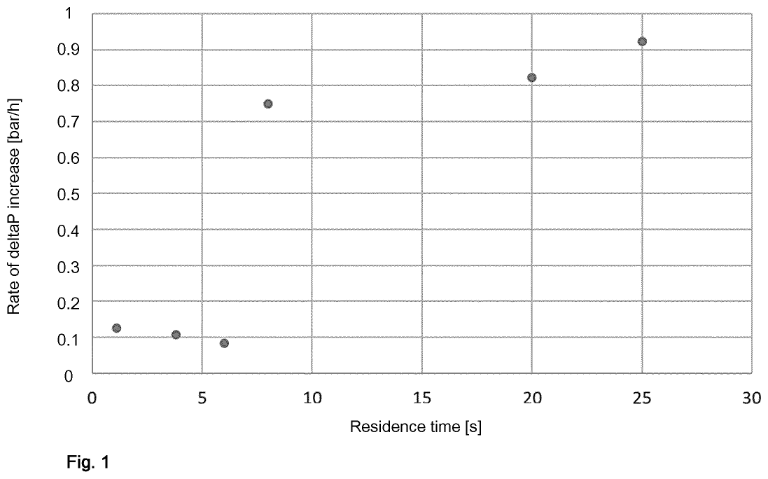

### CITED LITERATURE

EP 3 380 459 B1
Ullmann's Encyclopedia of Industrial Chemistry, 5^{th} edition, volume A 13 (1989) pages 443-466
EP 1 122 249 A1
EP 1 122 249 A1
WO 2015/049327 A1

1.-9. (canceled)

10. A process for the preparation of a liquid mixture comprising methanol, water and hydrogen peroxide, the process comprising
(i) providing a feed stream comprising methanol, and a feed stream comprising an aqueous hydrogen peroxide solution;
(ii) combining the feed stream comprising the methanol and the feed stream comprising the aqueous hydrogen peroxide solution provided according to (i) at a point in time t₁, so that a combined stream comprising methanol, hydrogen peroxide and water is obtained;
(iii) filtrating the combined stream obtained according to (ii) at a point in time t₂, through a filtration device, thereby obtaining a liquid mixture comprising methanol, hydrogen peroxide and water;
wherein the period of time between t₁ and t₂ is in the range of from 8 seconds to 1 hour.

11. The process for the preparation of a liquid mixture comprising methanol, water and hydrogen peroxide according to claim 10, further comprising
(iv) adding to the feed stream comprising methanol a further feed stream comprising propylene before (ii) or adding to the liquid mixture obtained in (iii) a further feed stream comprising propylene,
thereby obtaining a liquid mixture comprising methanol, hydrogen peroxide, water and propylene.

12. The process for the preparation of a liquid mixture comprising methanol, water and hydrogen peroxide according to claim 10, wherein the hydrogen peroxide is obtained from an anthraquinone process.

13. The process for the preparation of a liquid mixture comprising methanol, water and hydrogen peroxide according to claim 10, wherein the weight ratio of methanol:hydrogen peroxide (w/w) in the combined stream obtained in (ii) is in the range of from 15:1 to 5:1.

14. The process for the preparation of a liquid mixture comprising methanol, water and hydrogen peroxide according to claim 11, wherein the weight ratio of propylene:hydrogen peroxide (w/w) in the liquid mixture, which is obtained in (iv), is in the range of from 1:1 to 5:1;
and/or
wherein the weight ratio of methanol:hydrogen peroxide (w/w) in the liquid mixture, which is obtained in (iv), is in the range of from 15:1 to 5:1;
and/or
wherein the weight ratio of methanol:propylene (w/w) in the liquid mixture, which is obtained in (iv), is in the range of from 10:1 to 1:0.1.

15. The process for the preparation of a liquid mixture comprising methanol, water and hydrogen peroxide according to claim 10, wherein the filtration device according to (iii) is a filter selected from the group consisting of pleated filter, cuno type filter and mixed forms of these filter types.

16. A process for the preparation of propylene oxide which comprises utilizing a liquid mixture obtained from the process according to claim 10.

17. A method for the preparation of propylene oxide comprising
(i) providing a feed stream comprising methanol, a feed stream comprising an aqueous hydrogen peroxide solution and a feed stream comprising propylene;
(ii) combining the feed stream comprising the methanol and the feed stream comprising the aqueous hydrogen peroxide solution provided according to (i) at a point in time t₁, so that a combined stream comprising methanol, hydrogen peroxide and water is obtained;
(iii) filtrating the combined stream obtained according to (ii) at a point in time t₂, through a filtration device, thereby obtaining a liquid mixture comprising methanol, hydrogen peroxide and water;
(iv) combining the feed stream comprising propylene either with the combined stream obtained in (ii) or with the liquid mixture obtained in (iii), thereby obtaining a liquid mixture comprising methanol, hydrogen peroxide, water and propylene;
(v) bringing the liquid mixture obtained according to (iv) in an epoxidation zone in contact with an epoxidation catalyst comprising a zeolitic material having a framework structure comprising Si, O, and Ti under epoxidation reaction conditions, thereby obtaining in the epoxidation zone a mixture comprising propylene oxide, methanol and water,
wherein the period of time between t₁ and ta is in the range of from 8 seconds to 1 hour.

18. Propylene oxide obtained from the method according to claim 17.
